(19) United States
(12) Patent Application Publication
(10) Pub. No.: US 2022/0023096 A1
(43) Pub. Date: Jan. 27, 2022

(54) VITRECTOMY PROBE INCLUDING TISSUE MANIPULATION FEATURES AND METHOD OF MANUFACTURING A VITRECTOMY PROBE

(71) Applicant: Medical Instrument Development Laboratories, Inc., San Leandro, CA (US)

(72) Inventors: Erik W. Peterson, Walnut Creek, CA (US); Kai Chen, Potomac, MD (US); Yang Wei, San Mateo, CA (US)

(21) Appl. No.: 17/311,054

(22) PCT Filed: Nov. 25, 2019
(86) PCT No.: PCT/US2019/062971

§ 371 Date: Jun. 4, 2021

Related U.S. Application Data
(60) Provisional application No. 62/775,535, filed on Dec. 5, 2018.

(51) Int. Cl. A61F 9/007

(52) U.S. Cl. CPC A61F 9/00736

(57) ABSTRACT

A vitrectomy probe includes a hollow needle having a sidewall and a tip, a port formed in the sidewall of the hollow needle and spaced apart from the tip, and a cutter positioned within the hollow needle. The cutter is slidable relative to the port to cut tissue within the port. The vitrectomy probe also includes a manipulation feature defined by at least one recess formed in the tip of the hollow needle, the sidewall of the hollow needle, or both. The manipulation feature is configured to facilitate manipulation of tissue using the hollow needle.

### CROSS-REFERENCE TO RELATED APPLICATIONS

This application claims priority to U.S. Provisional Patent Application No. 62/775,535, filed Dec. 5, 2018, the entire contents of which are incorporated by reference herein.

### BACKGROUND

Embodiments relate to cutting instruments for ophthalmic surgery and, more particularly, to vitrectomy probes.

An ophthalmic cutting device is a surgical instrument for use in eye surgery. The cutting device is typically used to remove portions of the vitreous humor of the eye. Conventional cutting devices include two principal parts: a hollow needle having a cutting port, and a slidable cutter positioned within the needle. In use, the cutting device is inserted into an incision in the eye. As vitreous tissue enters the cutting port of the needle, the cutter slides past the port to cut the tissue. A vacuum may be applied to the cutter to remove cut tissue from the cutting device.

### SUMMARY

While performing an ophthalmic procedure, it may be beneficial to move or otherwise manipulate tissue in the eye. It is possible and usually preferable to use specific surgical tools to manipulate tissue. However, a surgeon may also simply try to move tissue with the instrument at hand. This is true when surgeons use vitrectomy probes even though such probes do not, in general, include features specifically designed for tissue manipulation. While some very limited tissue manipulation is possible using a standard vitrectomy probe, manipulation capability is crude and unrefined. Thus, improvements would be useful.

One embodiment provides a vitrectomy probe including a hollow needle having a sidewall and a tip, a port formed in the sidewall of the hollow needle and spaced apart from the tip, and a cutter positioned within the hollow needle. The cutter is slidable relative to the port to cut tissue within the port. The vitrectomy probe also includes a manipulation feature defined by at least one recess formed in the tip of the hollow needle, the sidewall of the hollow needle, or both. The manipulation feature is configured to facilitate manipulation of tissue using the hollow needle.

Another embodiment provides a method of manufacturing a vitrectomy probe. The method includes providing a hollow needle having a sidewall and a tip, and forming a port in the sidewall of the hollow needle. The port is spaced apart from the tip. The method also includes forming a recess in the tip of the hollow needle, the sidewall of the hollow needle, or both. The recess defines a manipulation feature configured to facilitate manipulation of tissue using the hollow needle.

Other aspects, examples, and embodiments will become apparent by consideration of the detailed description and accompanying drawings.

### BRIEF DESCRIPTION OF THE DRAWINGS

FIG. 1 illustrates an example of an ophthalmic surgical cutting apparatus including a vitrectomy probe.

FIG. 2 is an enlarged, cross-sectional view of a portion of the vitrectomy probe shown in FIG. 1 with a single blade cutter.

FIG. 3 is an enlarged, cross-sectional view of a portion of the vitrectomy probe shown in FIG. 1 with a bi-blade cutter.

FIG. 4 is a perspective view of a portion of the vitrectomy probe shown in FIG. 1 with features formed using a wire electrical discharging machining process.

FIG. 5 is a cross-sectional view of the portion of the vitrectomy probe shown in FIG. 4.

FIG. 6 is a perspective view of a portion of a vitrectomy probe in accordance with one variation.

FIG. 7 is a cross-sectional view of the portion of the vitrectomy probe shown in FIG. 6.

FIG. 8 is a perspective view of a portion of another vitrectomy probe in accordance with another variation.

FIG. 9 is a cross-sectional view of the portion of the vitrectomy probe shown in FIG. 8.

FIG. 10 is a perspective view of a portion of another vitrectomy probe in accordance with another variation.

FIG. 11 is a cross-sectional view of the portion of the vitrectomy probe shown in FIG. 10.

FIG. 12 is a first perspective view of a portion of a vitrectomy probe in accordance with another variation.

FIG. 13 is a second perspective view of the portion of the vitrectomy probe shown in FIG. 12.

FIG. 14 is a cross-sectional view of the portion of the vitrectomy probe shown in FIG. 12.

FIG. 15 is a perspective view of a portion of a vitrectomy probe in accordance with still another variation.

FIG. 16 is a cross-sectional view of the portion of the vitrectomy probe shown in FIG. 15.

FIG. 17 is a perspective view of a portion of a vitrectomy probe in accordance with yet still another variation.

FIG. 18 is a cross-sectional view of the portion of the vitrectomy probe shown in FIG. 1.

FIG. 19 is a perspective view of a portion of a vitrectomy probe in accordance with another variation.

FIG. 20 is a cross-sectional view of the portion of the vitrectomy probe shown in FIG. 19.

### DETAILED DESCRIPTION

Before any embodiments are explained in detail, it is to be understood that the embodiments and examples are not limited in their application to the details of construction and the arrangement of components set forth in the following description or illustrated in the following drawings. Other embodiments are possible and capable of being practiced or of being carried out in various ways.

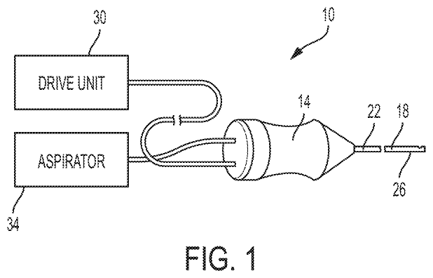

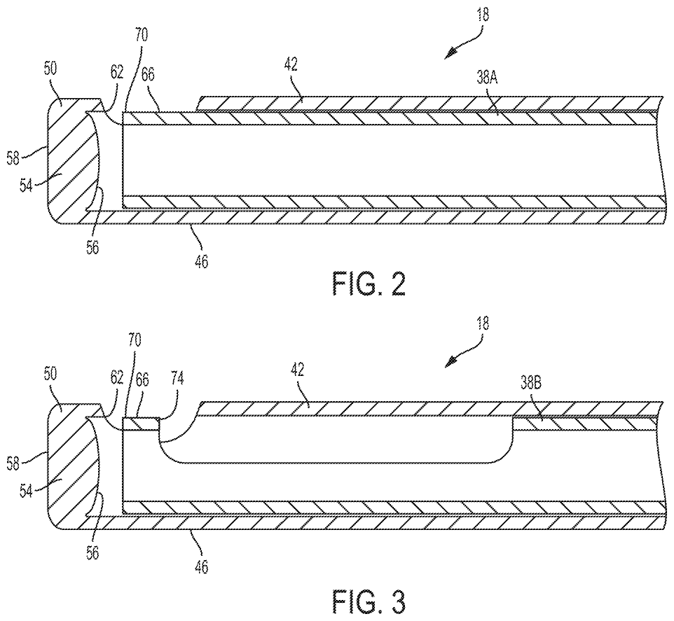

FIG. 1 illustrates an example cutting apparatus 10 for use in ophthalmic surgery. The illustrated apparatus 10 includes a handle 14 and a vitrectomy probe 18. The vitrectomy probe 18, or vitreous cutter, has a first end 22 connected to the handle 14 and a second end 26 opposite the first end 22. The cutting apparatus 10 also includes a drive unit 30 and an aspirator 34 (shown schematically) coupled to the handle 14. As further explained below, the drive unit 30 is connected to a cutter 38A, 38B (FIGS. 2 and 3) of the vitrectomy probe 18 to reciprocate the cutter 38. The aspirator 34 is fluidly coupled to the probe 18 to create a suction and remove cut pieces of tissue from the cutting apparatus 10.

As shown in FIGS. 2 and 3, the vitrectomy probe 18 includes a hollow needle 42 and the cutter 38A, 38B. The needle 42 is generally cylindrical and is typically size 20 gauge or smaller. The needle 42 includes a sidewall 46 defining an outer surface of the probe 18. The needle 42 also includes a tip 50 formed at the second or distal end 26 of the probe 18. In the illustrated example, the tip 50 is formed by a weld bead 54. The weld bead 54 is formed by melting an end of the needle 42 to create a closed tip. In the illustrated embodiment, the weld bead 54 is generally spherical, with a convex surface 56 facing toward an interior of the hollow needle 42. The weld bead 54 also has a generally planar surface 58 facing outwardly from the needle 42. The generally planar surface 58 may be formed by, for example, grinding or otherwise machining the tip 50 to form a flat surface and remove excess material. In other examples, the tip 50 may be formed by a separate piece of material, such as an end cap, that is brazed, welded, glued, molded, or otherwise secured to the end of the needle 42.

A port 62 is formed in the sidewall 46 of the needle 42. The port 62 is positioned near, but spaced apart from the tip 50. The port 62 extends through the sidewall 46 to provide fluid communication to the interior of the needle 42. The port 62 is configured to receive tissue (for example, a portion of the vitreous) during operation of the cutting apparatus 10. As a consequence, the port 62 may also be referred to as a cutting port and/or an aspiration port.

The cutter 38A, 38B is positioned within the hollow needle 42. In the illustrated examples, the cutter 38A, 38B is generally cylindrical and hollow. The cutter 38A, 38B includes a first cutter end connected to the drive unit 30 (FIG. 1) and a second cutter end 66 opposite the first cutter end. In the example shown in FIG. 2, the cutter end 66 of the cutter 38A defines a single cutting edge 70. The cutter 38A may be referred to as a single blade cutter. In the example shown in FIG. 3, the second cutter end 66 of the cutter 38B defines two cutting edges 70, 74. The cutter 38B may be referred to as a bi-blade cutter.

During use, the drive unit 30 is configured to linearly reciprocate the cutter 38A, 38B within the needle 42. As the cutter 38A, 38B reciprocates, the cutter 38A, 38B moves relative to the port 62. More particularly, the cutting edge(s) 70, 74 of the cutter 38A, 38B reciprocate(s) back-and-forth across the port 62. The cutting edge 70 of the single blade cutter 38A (FIG. 2) cooperates with an edge of the sidewall 46 defining the port 62 as the cutter 38A moves in one direction (to the left in the drawing) to cut (for example, shear) tissue extending into the port 62. The cutting edges 70, 74 of the bi-blade cutter 38B (FIG. 3) also cooperate with the edge of the sidewall 46 defining the port to cut (for example, shear) tissue extending into the port 62. With the bi-blade cutter 38B, however, one of the cutting edges 70 cuts tissue as the cutter 38B moves past the port in a first direction (to the left in the drawing), while the other cutting edge 74 cuts tissue as the cutter 38B moves past the port in a second direction (to the right in the drawing). In both examples, suction created by the aspirator 34 (FIG. 1) helps pull the tissue into the port 62 prior to each cut.

The port 62 may be formed in the needle 42 by various processes. One such process is a wire electrical discharge machining (EDM) process. During such a process, the needle 42 is moved against a wire carrying a voltage to remove material from the needle 42. When the needle 42 is sufficiently close to the wire, current from the wire vaporizes material on the needle 42. Further movement of the needle 42 relative to the wire continues the vaporization of material along the path of travel, forming the port 62.

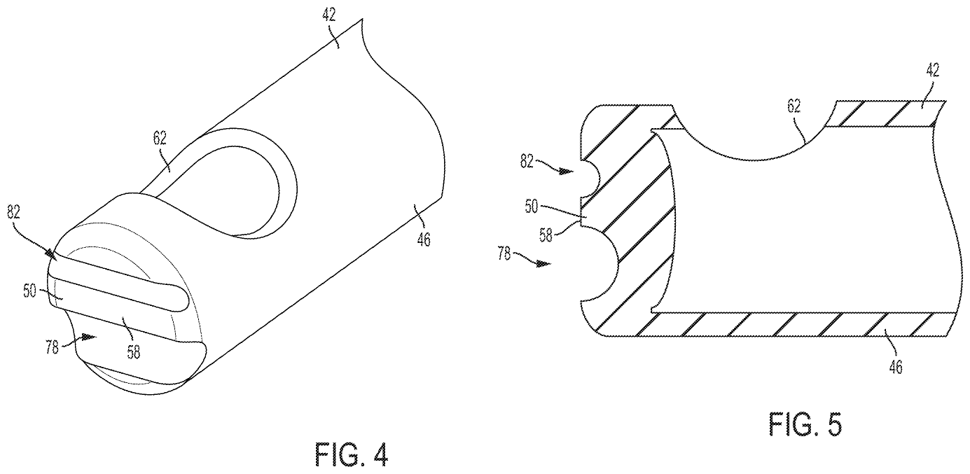

FIGS. 4 and 5 illustrate how the wire EDM process can be used to form additional features in the tip 50 of the needle 42. Recesses 78 and 82 have been formed by pressing the tip 50 of the needle 42 against the wire in an axial direction, with no lateral movement. In this example, the radius of the recess corresponds to the radius of the wire. Recess 78 illustrates the sort of feature which can be formed with a wire of 6 mils diameter, which is a commonly used wire size in the wire EDM process. Recess 82 illustrates the sort of feature which can be formed with a wire of 3 mils diameter, which is the smallest practical wire size for the wire EDM process. The needle size in these illustrations is 25 gauge (20 mils diameter), which is a commonly used size for vitrectomy probes.

In general, the wire EDM process is capable of forming features with a radius greater than or equal to the radius of the wire. In FIGS. 4 and 5, the port 62 could be formed with either the 6 mil wire or the 3 mil wire by combining axial and lateral movements of the needle 42.

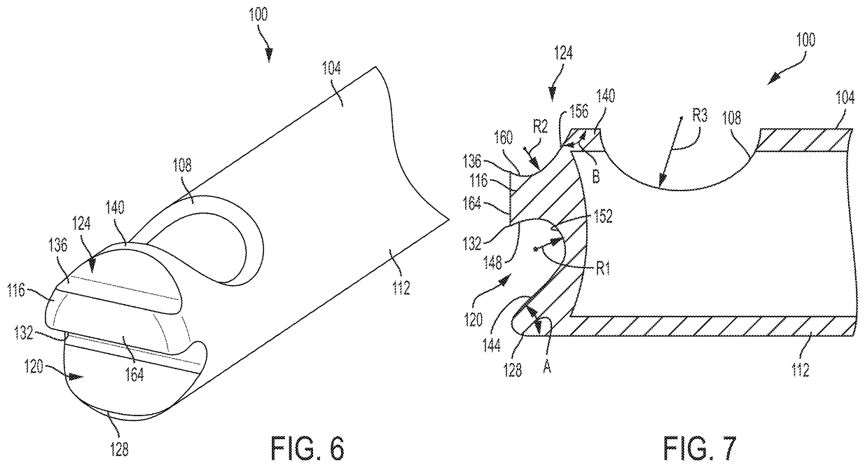

FIGS. 6 through 11 illustrate various useful features which could be formed in a tip of a needle by a wire EDM process. The features shown can be quickly and easily machined in the needle at little to no extra cost because the features are formed in the same plane as the port. FIGS. 4 and 9 illustrate features which could be formed with the more commonly used 6 mil wire. FIGS. 6 through 9 illustrate features which could be formed with the smaller 3 mil wire.

FIGS. 6 and 7 illustrate another vitrectomy probe 100. The probe 100 is similar to the probe 10 described above and includes a needle 104, a port 108, and a cutter. The needle 104 includes a sidewall 112 and a tip 116. In the illustrated example, the cutter is omitted to simplify the drawings, but may be the single blade cutter 38A (FIG. 2), the bi-blade cutter 38B (FIG. 3), or any other suitable cutter. Other features and variations of the probe 10 described above, but not specifically identified below may be included in the probe 100.

The illustrated probe 100 also includes one or more manipulation features formed in the needle 104. The manipulation features are configured to engage and manipulate tissue (for example, to pull, move, massage, or otherwise loosen portions of the vitreous) prior to cutting and are sometimes referred to as tissue manipulation features. In the illustrated example, the probe 100 includes a plurality of manipulation features defined by a first recess 120 and a second recess 124. More particularly, the illustrated probe 100 includes four manipulation features: a first manipulation feature 128, a second manipulation feature 132, a third manipulation feature 136, and a fourth manipulation feature 140. The manipulation features 128, 132, 136, 140 are different from each other. They provide different levels or degrees of tissue manipulation and may be more suitable for some applications or surgical procedures than other applications and procedures. Although the illustrated probe 100 includes four manipulation features, in other examples, the probe 100 may include a single manipulation feature or may include a subset of the manipulation features.

The first recess 120 is an arcuate recess formed generally in a central area of the tip 116. Unlike the port 108, the first recess 120 does not extend entirely through the tip 116 to the interior of the needle 104. Rather, the first recess 120 is a depression formed in the tip 116. The illustrated first recess 120 is defined by a first planar wall 144, a second planar wall 148 opposite the first planar wall 144, and a curved base 152 connecting the first and second planar walls 144, 148. The first planar wall 144 forms an acute angle A with an adjacent outer surface of the sidewall 112. In some embodiments, the acute angle A may be between about 15 degrees and about 60 degrees. In the illustrated example, the acute angle A is about 45 degrees. The base 152 has a radius of curvature R1. In the illustrated examples, the radius of curvature R1 is 3 mils.

The second recess 124 is an arcuate recess formed near an edge area of the tip 116 adjacent the port 108. Similar to the first recess 120, the second recess 124 does not extend entirely through the tip 116 or the sidewall 112 to the interior of the needle 104. Rather, the second recess 124 is a depression formed in the tip 116 and the sidewall 112. The second recess 124 is spaced apart from the port 108 and defined by a planar wall 156 and a curved base 160. The planar wall 156 forms an obtuse angle B with an adjacent outer surface of the sidewall 112. In some embodiments, the obtuse angle B may be between about 110 degrees and 160 degrees. In the illustrated example, the obtuse angle B is about 130 degrees. The base 160 has a radius of curvature R2. In the illustrated examples, the radius of curvature R2 is 3 mils.

The radius of curvature R1 of the first recess 120 and the radius of curvature R2 of the second recess 124 are formed in the same plane as a radius of curvature R3 of the port 108 (i.e., in the cross-sectional plane of FIG. 7). As such, the recesses 120, 124 and the port 108 may be formed in rapid succession using the same manufacturing process (for example, a wire electrical discharge machining process).

The illustrated first manipulation feature 128 is a pick defined by the first recess 120. More particularly, the pick 128 is defined between an outer surface of the sidewall 112 opposite from the port 108 and the first planar wall 144 of the first recess 120. The pick 128 allows a user to push or pick at tissue, thereby loosening a section of tissue for cutting.

The illustrated second manipulation feature 132 is a hook defined by the first recess 120. The hook is defined at an intersection between the second planar wall 148 of the first recess 120 and a planar surface 164 of the tip 116. By rotating the probe 100 ninety degrees in either direction so the pick 128 is moved away from the tissue, corners of the hook 132 are brought adjacent to the tissue so the user can push or pull at the tissue by engaging the tissue with the one of the corners.

The illustrated third manipulation feature 136 is a scraper defined by the second recess 124. More particularly, the scraper 136 is defined at an intersection between the curved base 160 of the second recess 124 and the planar surface 164 of the tip 116. By rotating the probe 100 one hundred eighty degrees so the port 108 is moved adjacent and facing the tissue, the scraper 136 can be moved across the tissue to scrape and loosen the tissue. Corners of the scraper 136 may also be used as hooks, similar to the corners of the hook 132.

The illustrated fourth manipulation feature 140 is a ridge defined by the second recess 124. The ridge 140 is defined between the planar wall 156 of the second recess 124 and the port 108. By rotating the probe 100 one hundred eighty degrees so the port 108 is moved adjacent and facing the tissue (similar to when using the scraper 136), the ridge 140 can engage the tissue to push on and massage the tissue.

During use, a surgeon may push against tissue (for example, the vitreous membrane of an eye) using any of the manipulation features 128, 132, 136, 140. Different manipulation features 128, 132, 136, 140 may be used depending on the type of activity (for example, push, pull, scrape, etc.) desired by the surgeon, the level of control desired by the surgeon, and the orientation of the probe. Once a section of tissue is sufficiently loosened, moved, or otherwise manipulated by one of the manipulation features 128, 132, 136, 140, that piece of tissue is then drawn into the port 108 by creating a suction with the aspirator 34 (FIG. 1). The cutter (for example, the single blade cutter 38A or the bi-blade cutter 38B) is then reciprocated by the drive unit 30 (FIG. 1) to cut the tissue, which is pulled through and removed from the needle 104 by the aspirator 34.

The manipulation features 128, 132, 136, 140 allow a surgeon to manipulate tissue during complex procedures with a single tool, rather than having to use separate tools. Some surgeons have used the cutting port of a vitrectomy probe (with the aspirator creating a suction) to pull on tissue. The manipulation features 128, 132, 136, 140 described above provide more control over the manipulation of tissue compared to the relatively large cutting port.

The illustrated manipulation features 128, 132, 136, 140 are, preferably, integrally formed in the needle 104 by removing material from the needle 104. In other words, extra material is not added to the needle 104 to form the manipulation features 128, 132, 136, 140. This allows the needle 104 to still pass through cannulas used in modern, small-incision surgery. The weld bead (see the weld bead 54 in FIGS. 2 and 3) provides a substantial amount of material at the tip 116 into which the manipulation features 128, 132, 136, 140 can be formed without sacrificing the structural integrity of the needle 104.

The manipulation features 128, 132, 136, 140 can be formed in the needle 104 at the same time and using the same process as the port 108. In some examples, the port 108 is formed in the needle 104 using a wire electrical discharge machining (EDM) process. In such examples, the manipulation features 128, 132, 136, 140 are formed in the tip 116 of the needle 104 using the same process before or after the port 108 is formed, thereby providing almost no increased costs to the manufacturing process. In other examples, other suitable processes, such as laser cutting, may be used for forming the port 108 and/or the manipulation features 128, 132, 136, 140. Additionally or alternatively, the manipulation features 128, 132, 134, 140 can be formed in a different plane from the port 108.

The vitrectomy probe 100 can be manufactured by first providing the hollow needle 104 (i.e., a section of hollow feed stock that is cut to a desired length). One end of the hollow needle 104 is then closed. In some examples, the end is closed by forming a weld bead at the end. The weld bead is formed by melting the end of the needle 104. After the end is melted, the end can be ground or otherwise machined to remove excess material from the tip 116. Grinding the tip 116 also forms the planar surface 164. In other examples, the end is closed by securing an end cap or other suitable piece of material to the tip 116.

The port 108 and the manipulation features 128, 132, 136, 140 are then formed in the needle 104. As noted above, in some examples, the port 108 and the manipulation features 128, 132, 136, 140 are formed using the wire EDM process. During such a process, the needle 104 is moved against a wire carrying a voltage to remove material from the needle 104. When the needle 104 is sufficiently close to the wire, current from the wire vaporizes material on the needle 104, forming the port 108 and the recesses 120, 124 that define the manipulation features 128, 132, 136, 140. In some examples, the wire can have a diameter of 6 mils to create recesses having diameters of 6 mils or greater. In other examples, the wire can have a diameter of 3 mils to create recesses having diameters of 3 mils or greater. By forming the recesses 120, 124 so their radii of curvature R1, R2 are in the same plane as the radius of curvature R3 of the port 108, the recesses 120, 124 can be quickly and easily machined in the needle 104 at little to no extra cost because the needle 104 does not need to be rotated about its length after forming the port 108 before being brought close to the wire again.

After the port 108 and the recesses 120, 124 are formed in the needle 104, a cutter, such as the single blade cutter 38A (FIG. 2) or the bi-blade cutter 38B (FIG. 3), is positioned in the needle 104. The cutter is inserted through an open end of the needle 104 (i.e., the end opposite the closed tip). The handle 14, the drive unit 30, and the aspirator 34 are then connected to the needle 104 and the cutter to complete assembly of the probe 100.

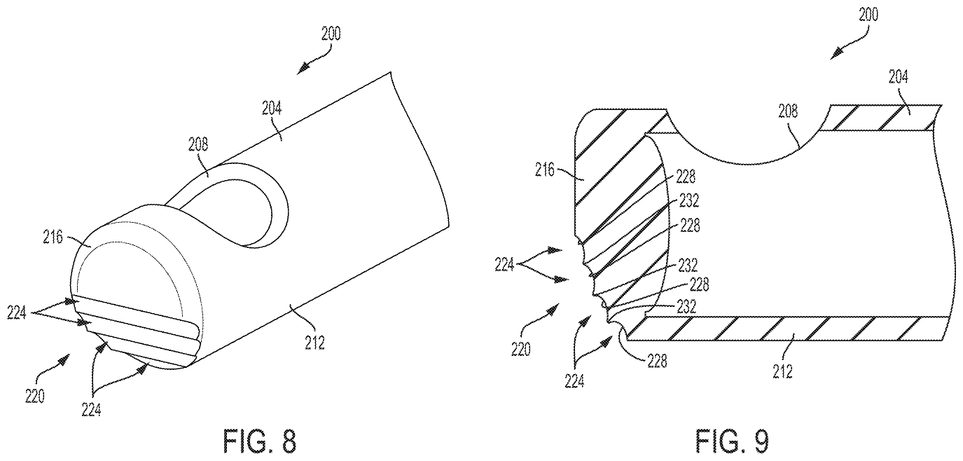

FIGS. 8 and 9 illustrate another vitrectomy probe 200. The probe 200 is similar to the probes 10, 100 described above and includes a needle 204, a port 208, and a cutter. The needle 204 includes a sidewall 212 and a tip 216. In the illustrated example, the cutter is omitted to simplify the drawings, but may be the single blade cutter 38A (FIG. 2), the bi-blade cutter 38B (FIG. 3), or other suitable cutter. Other features and variations of the probes 10, 100 described above, but not specifically identified below may be included in the probe 200.

Similar to the probe 100, the illustrated probe 200 includes a manipulation feature 220 formed in the tip 216. The manipulation feature 220 is defined by a series of adjacent recesses 224. In the illustrated example, the manipulation feature 220 is defined by four recesses 224. In other examples, the manipulation feature 220 may be formed by fewer or more recesses 224. The recesses 224 are formed near an edge area of the tip 216 opposite from the port 208. The recesses 224 are arcuate recesses having curved bases 228. The curved bases 228 intersect to form peaks 232, giving part of the tip 216 a wave-like profile (as viewed in FIG. 7). In other words, the recesses 224 form a corrugated surface that can be used to massage and loosen tissue prior to cutting.

The recesses 224 may be formed using a wire EDM process, similar to the recesses 120, 124 of the probe 100.

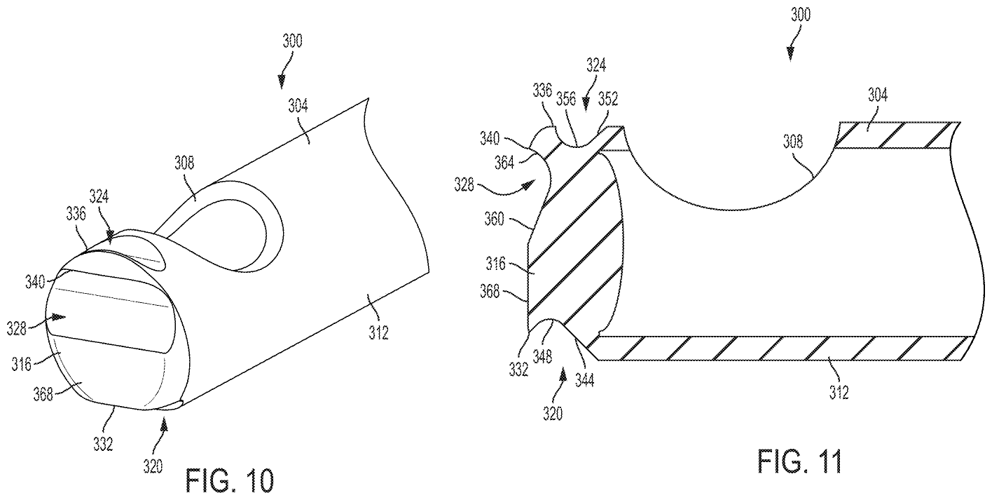

FIGS. 10 and 11 illustrate another vitrectomy probe 300. The probe 300 is similar to the probes 10, 100 described above and includes a needle 304, a port 308, and a cutter. The needle 304 includes a sidewall 312 and a tip 316. In the illustrated example, the cutter is omitted to simplify the drawings, but may be the single blade cutter 38A (FIG. 2), the bi-blade cutter 38B (FIG. 3), or any other suitable cutter. Other features and variations of the probes 10, 100 described above, but not specifically identified below may be included in the probe 300.

Similar to the probe 100, the illustrated probe 300 includes manipulation features formed in the tip. In the illustrated example, the probe 300 includes three recesses 320, 324, 328 defining three manipulation features: a first manipulation feature 332, a second manipulation feature 336, and a third manipulation feature 340. The first recess 320 is formed in an edge area of the tip 316 opposite from the port 308. The first recess 320 is an arcuate recess defined by a planar wall 344 and a curved base 348. The second recess 324 is formed in an edge area of the tip 316 adjacent the port 308. The second recess 324 is an arcuate recess defined by a planar wall 352 and a curved base 356. The third recess 328 is formed in a central area of the tip 316 between the first recess 320 and the second recess 324. The third recess 328 is an arcuate recess defined by a planar wall 360 and a curved base 364.

The first manipulation feature 332 is a scraper defined by the first recess 320. More particularly, the scraper 332 is defined by an intersection between the curved base 348 of the first recess 320 and a planar surface 368 of the tip 316.

The second manipulation feature 336 is a hook defined by the second recess 324. More particularly, the hook 336 is defined by an intersection between the curved base 356 of the second recess 324 and an outer surface of the sidewall 312 adjacent the port 308.

The third manipulation feature 340 is a pick defined by the third recess 328. More particularly, the pick 340 is defined by an intersection between the curved base 364 of the third recess 328 and the planar surface 368 of the tip 316.

The recesses 320, 324, 328 may be formed using a wire EDM process or a laser cut process, similar to the recesses 120, 124 of the probe 100.

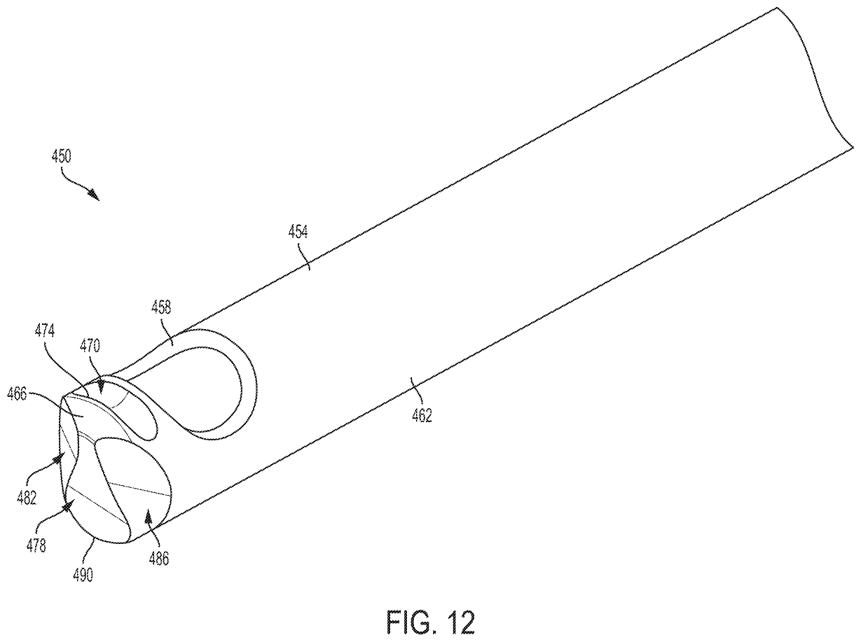

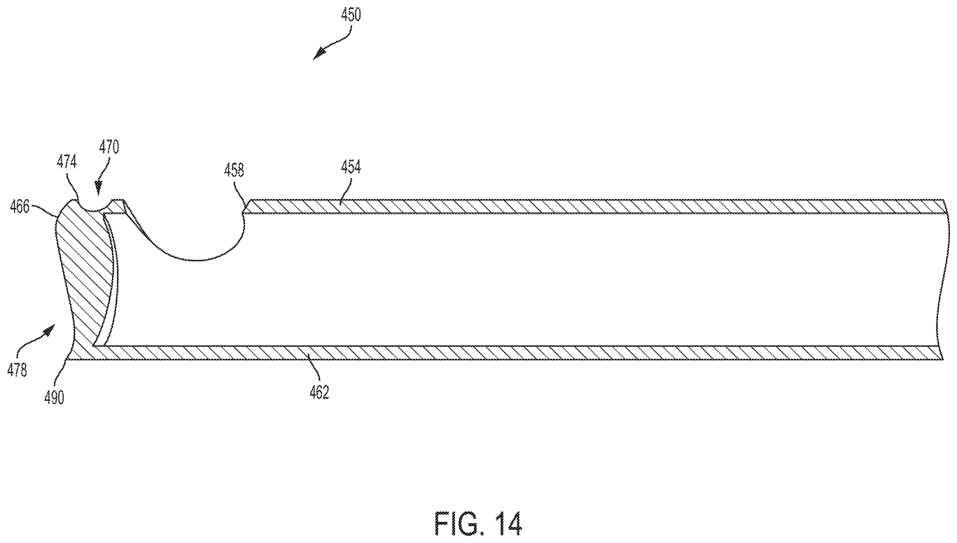

FIGS. 12-14 illustrate another vitrectomy probe 450. The probe 450 is similar to the probes 10, 110 described above and includes a needle 454, a port 458, and a cutter. The needle 454 includes a sidewall 462 and a tip 466. In the illustrated example, the cutter is omitted to simply the drawings, but may be the single blade cutter 38A (FIG. 2), the bi-blade cutter 38B (FIG. 3), or any other suitable cutter. Other features and variations of the probes 10, 100 described above, but not specifically identified below may be included in the probe 450.

Similar to the probe 100, the illustrated probe 450 includes manipulation features formed in the tip 466. In the illustrated embodiment, the probe 450 includes one recess 470 defining a first manipulation feature 474 and three recesses 478, 482, 486 defining a second manipulation feature 490. The first recess 470 is formed in an edge area of the tip 466 adjacent the port 458 and creates a hook (i.e., the first manipulation feature 474) adjacent the port 458. The second recess 478 is formed on an end surface of the tip 466 that is perpendicular to the sidewall 462. The third and fourth recesses 482, 486 are formed on opposing sides of the second recess 478. Together, the second, third, and fourth recesses 478, 482, 486 define a pick (i.e., the second manipulation feature 490).

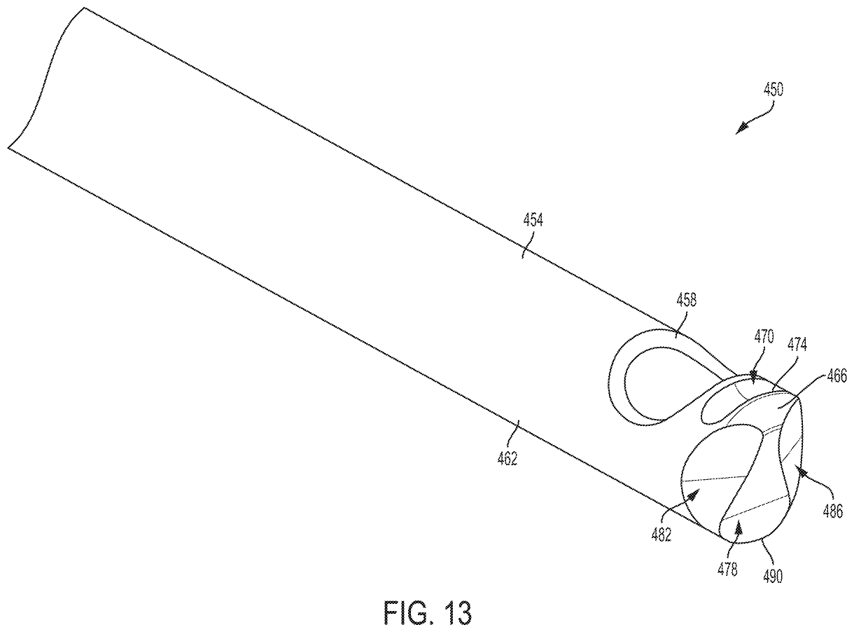

In the embodiment of FIGS. 12 and 13, the recesses 470, 478, 482, 486 may be formed by a laser cutting process or by a combination or laser cutting and EDM processes. For example, the first recess 470 may be formed with the port 458 by an EDM process, while the second, third, and fourth recesses 478, 482, 486 may be formed by laser cutting. In addition, at least some of the recesses (e.g., the third and fourth recesses 482, 486) are formed in planes different than the plane in which the port 458 is formed.

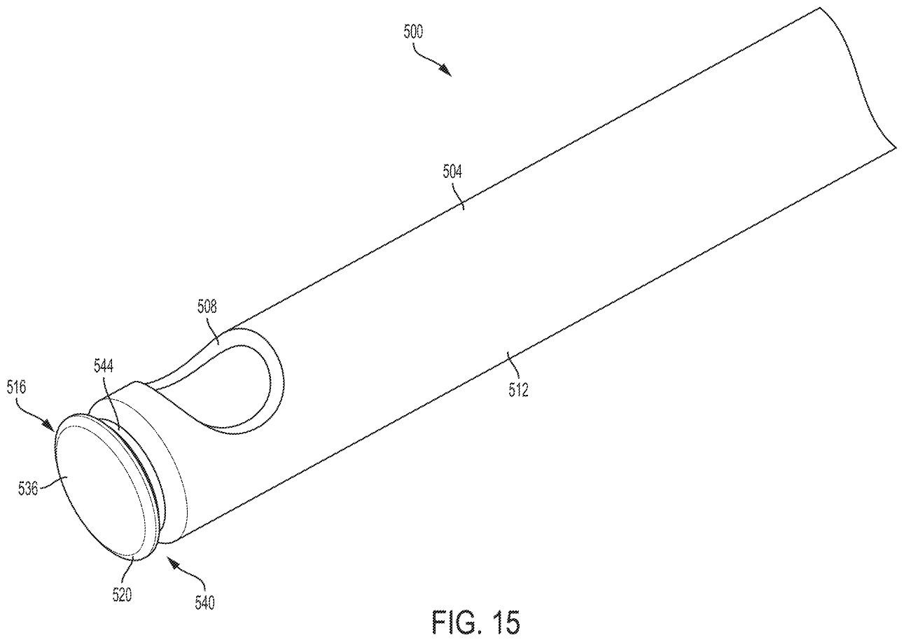

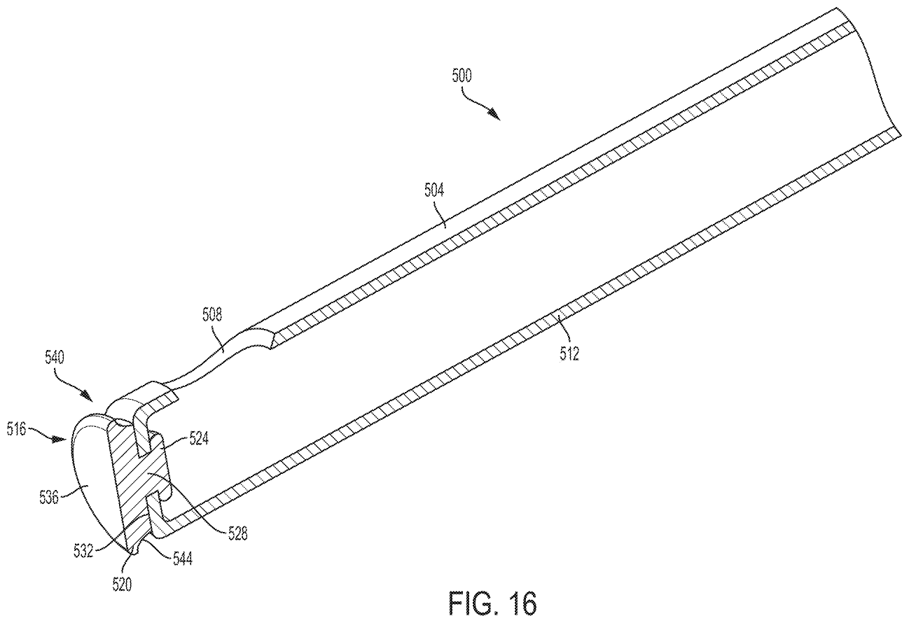

FIGS. 15 and 16 illustrate another vitrectomy probe 500. The probe 500 is similar to the probes 10, 100 described above and includes a needle 504, a port 508, and a cutter. The needle 504 includes a sidewall 512 and a tip 516. In the illustrated example, the tip 516 is defined by a separate end cap 520 that is secured to the sidewall 512. The illustrated end cap 520 includes an enlarged boss 524 positioned inside the needle 504, a neck 528 extending through an opening in an end wall 532 of the needle 504, and an outer disk 536 positioned outside the needle 504. In the illustrated example, the cutter is omitted to simplify the drawings, but may be the single blade cutter 38A (FIG. 2), the bi-blade cutter 38B (FIG. 3), or any other suitable cutter. Other features and variations of the probes 10, 100 described above, but not specifically identified below may be included in the probe 500.

Similar to the probe 100, the illustrated probe 500 includes a manipulation feature 540 formed in the tip 516. The illustrated manipulation feature 540 is defined by an annular recess 544 formed between the outer disk 536 of the end cap 520 and the end wall 532 of the needle 504. The annular recess 544 creates a hook around the entire circumference of the tip 516. The annular recess 544 may be formed before or after the end cap 520 is secured to the sidewall 512

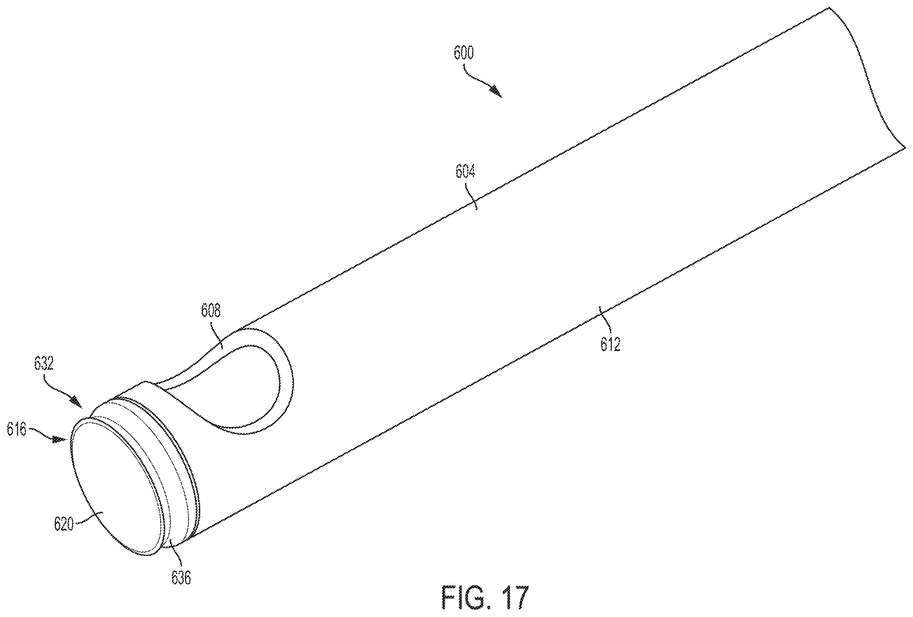

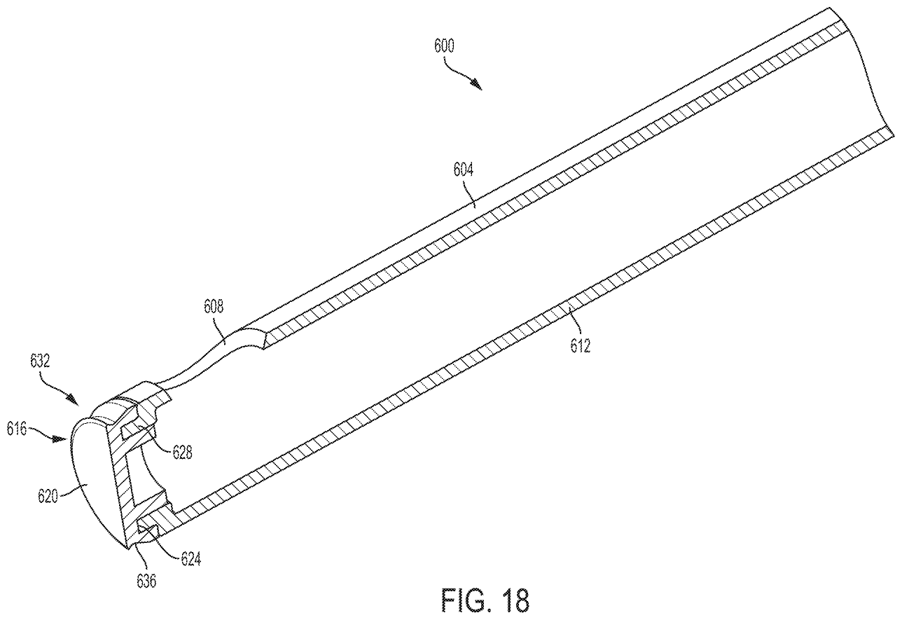

FIGS. 17 and 18 illustrate another vitrectomy probe 600. The probe 600 is similar to the probes 10, 100 described above and includes a needle 604, a port 608, and a cutter. The needle 604 includes a sidewall 612 and a tip 616. In the illustrated example, the tip 616 is defined by a separate end cap 620 that is secured to the sidewall 612. The illustrated end cap 620 includes an annular groove 624 that receives an annular projecting portion 628 of the sidewall 612. The end cap 620 is then held in place by, for example, brazing, welding, molding, and/or press-fitting. In the illustrated example, the cutter is omitted to simplify the drawings, but may be the single blade cutter 38A (FIG. 2), the bi-blade cutter 38B (FIG. 3), or any other suitable cutter. Other features and variations of the probes 10, 100 described above, but not specifically identified below may be included in the probe 600.

Similar to the probe 100, the illustrated probe 600 includes a manipulation feature 632 formed in the tip 616. The illustrated manipulation feature 632 includes an annular recess 636 formed in the end cap 620. The annular recess 636 creates a relatively sharp pick around the entire circumference of the tip 616. The annular recess 636 may be formed before or after the end cap 620 is secured to the sidewall 612.

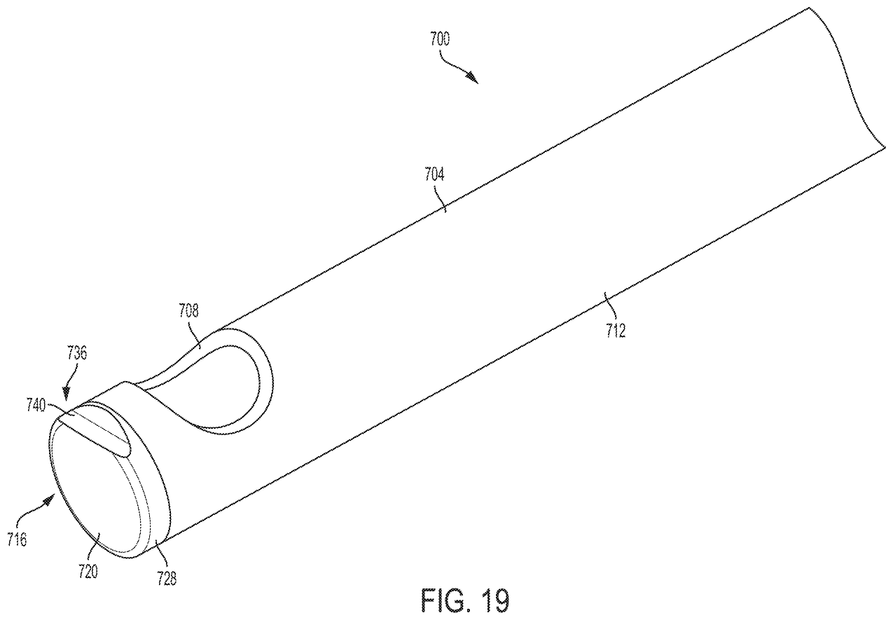

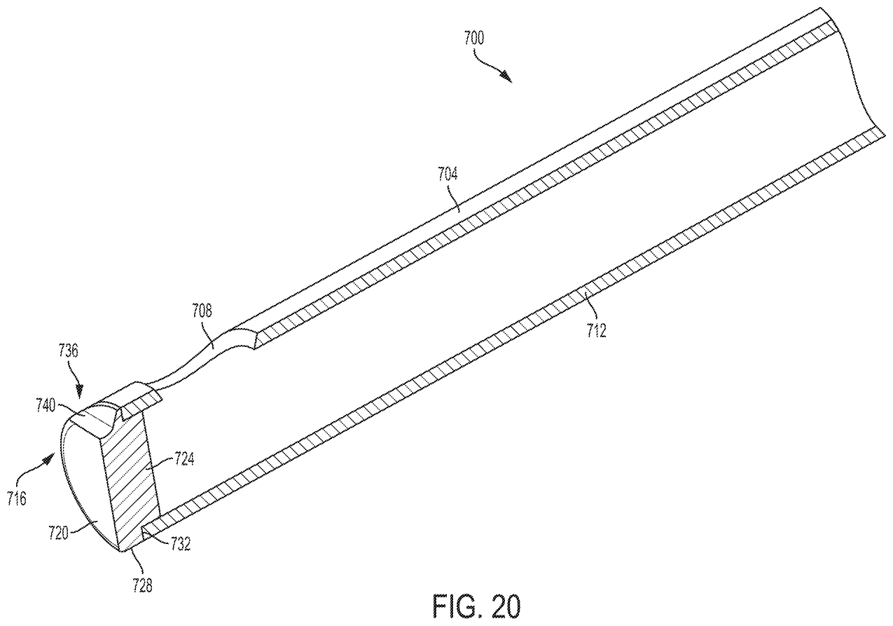

FIGS. 19 and 20 illustrate another vitrectomy probe 700. The probe 700 is similar to the probes 10, 100 described above and includes a needle 704, a port 708, and a cutter. The needle 704 includes a sidewall 712 and a tip 716. In the illustrated example, the tip 716 is defined by a separate end cap 720 that is secured to the sidewall 712. The illustrated end cap 720 includes a small diameter section 724 positioned inside the sidewall 712 and a large diameter section 728 positioned outside the sidewall 712. A shoulder 732 is formed between the small diameter section 724 and the large diameter section 728 and abuts an end of the sidewall 712. The small diameter section 724 has an outer diameter that generally matches an inner diameter of the sidewall 712 such that the end cap 720 tightly engages the sidewall 712. The large diameter section 728 has an outer diameter that generally matches an outer diameter of the sidewall 712. In the illustrated example, the cutter is omitted to simplify the drawings, but may be the single blade cutter 38A (FIG. 2), the bi-blade cutter 38B (FIG. 3), or any other suitable cutter. Other features and variations of the probes 10, 100 described above, but not specifically identified below may be included in the probe 700.

Similar to the probe 100, the illustrated probe 700 includes a manipulation feature 736 formed in the tip 716. The illustrated manipulation feature 736 includes a recess 740 formed in a side of the end cap 720 adjacent the port 708. The recess 740 may be formed before or after the end cap 720 is secured to the sidewall 712.

The above disclosure provides a vitrectomy probe having one or more tissue manipulation features. Although the vitrectomy probes are described above as separate examples, features of the probes may be interchanged or used in combination with each other. Various features, advantages, and embodiments are set forth in the following claims.

What is claimed is:

1. A vitrectomy probe comprising:
a hollow needle having a sidewall and a tip;
a port formed in the sidewall of the hollow needle and spaced apart from the tip;
a cutter positioned within the hollow needle, the cutter being slidable relative to the port to cut tissue within the port; and
a manipulation feature defined by at least one recess formed in the tip of the hollow needle, the sidewall of the hollow needle, or both, the manipulation feature configured to facilitate manipulation of tissue using the hollow needle.

2. The vitrectomy probe of claim 1, wherein the at least one recess is an arcuate recess having a curved base.

3. The vitrectomy probe of claim 2, wherein the port has a first radius of curvature and the arcuate recess has a second radius of curvature, and wherein the first and second radii of curvature are located in a same plane.

4. The vitrectomy probe of claim 2, wherein the port has a first radius of curvature and the arcuate recess has a second radius of curvature, and wherein the first and second radii of curvature are located in different planes.

5. The vitrectomy probe of claim 2, wherein the arcuate recess has a radius of curvature that is 3 mils or less.

6. The vitrectomy probe of claim 1, wherein the tip of the hollow needle is defined by a weld bead, and wherein the at least one recess is formed in the weld bead.

7. The vitrectomy probe of claim 1, wherein the tip of the hollow needle is defined by an end cap that is separate from and secured to the sidewall.

8. The vitrectomy probe of claim 7, wherein the at least one recess is defined between the end cap and the sidewall.

9. The vitrectomy probe of claim 7, wherein the at least one recess is formed in the end cap.

10. The vitrectomy probe of claim 1, wherein the at least one recess is an annular recess formed around an entire circumference of the tip.

11. The vitrectomy probe of claim 1, wherein the cutter includes a first cutting edge and a second cutting edge spaced apart from the first cutting edge, wherein the first cutting edge is configured to cut tissue as the cutter moves past the port in a first direction, and wherein the second cutting edge is configured to cut tissue as the cutter moves past the port in a second direction that is opposite the first direction.

12. The vitrectomy probe of claim 1, wherein the manipulation feature includes one or more selected from the group consisting of a pick, a hook, a scraper, or a corrugated surface at least partially defined by the at least one recess.

13. The vitrectomy probe of claim 12, wherein the manipulation feature is a pick defined at an intersection of the tip of the hollow needle and the sidewall of the hollow needle by one or more recesses in the tip of the hollow needle.

14. The vitrectomy probe of claim 12, wherein the manipulation feature is a hook defined by a recess in the sidewall of the hollow needle.

15. The vitrectomy probe of claim 12, wherein the manipulation feature is a scraper defined in the tip of the hollow needle by the at least one recess encompassing an intersection of the tip of the hollow needle and the sidewall of the hollow needle.

16. The vitrectomy probe of claim 12, wherein the manipulation feature is a corrugated surface defined by a plurality of recesses in the tip of the hollow needle.

17. The vitrectomy probe of claim 1, wherein the manipulation feature is a first manipulation feature and the at least one recess is a first recess, and further comprising a second manipulation feature spaced apart from the first manipulation feature and defined by a second recess formed in the tip of the hollow needle, the sidewall of the hollow needle, or both.

18. The vitrectomy probe of claim 17, wherein the second manipulation feature is different than the first manipulation feature.

19. A method of manufacturing a vitrectomy probe, the method comprising:
providing a hollow needle having a sidewall and a tip;
forming a port in the sidewall of the hollow needle, the port being spaced apart from the tip; and
forming a recess in the tip of the hollow needle, the sidewall of the hollow needle, or both, the recess defining a manipulation feature configured to facilitate manipulation of tissue using the hollow needle.

20. The method of claim 19, wherein forming the recess includes forming an arcuate recess in the tip, the sidewall, or both, the arcuate recess having a curved base.

21. The method of claim 20, wherein forming the port includes forming the port with a first radius of curvature in a plane, and wherein forming the arcuate recess includes forming the arcuate recess with a second radius of curvature in the plane.

22. The method of claim 20, wherein forming the port includes forming the port with a first radius of curvature in a plane, and wherein forming the arcuate recess includes forming the arcuate recess with a second radius of curvature in another plane.

23. The method of claim 19, wherein forming the port includes forming the port in the sidewall of the hollow needle using a wire electrical discharge machining process, and wherein forming the recess includes forming the recess using the wire electrical discharge machining process or a laser cutting process.

24. The method of claim 19, further comprising forming a weld bead at the tip of the hollow needle prior to forming the recess.

25. The method of claim 24, wherein forming the weld bead includes melting an end of the hollow needle to create a generally spherical, closed tip.

26. The method of claim 19, further comprising securing an end cap to the sidewall of the hollow needle to form the tip.

27. The method of claim 19, wherein forming the recess includes forming the recess to define one or more selected from the group consisting of a pick, a hook, a scraper, or a corrugated surface.

28. The method of claim 19, wherein the recess is a first recess and the manipulation feature is a first manipulation feature, and further comprising forming a second recess in the tip of the hollow needle, the sidewall of the hollow needle, or both, the second recess being spaced apart from the first recess and defining a second manipulation feature.

29. The method of claim 19, wherein the port and the recess are formed by an electrical discharge machining (EDM) process.

30. The method of claim 19, wherein the port and the recess are formed by a laser cutting process.
